(19) United States
(12) Patent Application Publication
(10) Pub. No.: US 2010/0317023 A1
(43) Pub. Date: Dec. 16, 2010

(54) BIOCHEMICAL MARKERS FOR CVD RISK ASSESSMENT

(75) Inventors: Per Qvist, Klampenborg (DK); Anne-Christine B. Jensen, Copenhagen S (DK); Bijue Wang, Beijing City (CN); Natasha Barascuk, Copenhagen Ø (DK)

Correspondence Address: EDWARDS ANGELL PALMER & DODGE LLP, P.O. BOX 55874, BOSTON, MA, 02205, US

(73) Assignee: NORDIC BIOSCIENCE A/S, Herlev (DK)

(21) Appl. No.: 12/794,808

(22) Filed: Jun. 7, 2010

Related U.S. Application Data
(60) Provisional application No. 61/268,224, filed on Jun. 9, 2009.

(51) Int. Cl. G01N 33/53

(52) U.S. Cl. 435/7.1

(57) ABSTRACT

A method of diagnosis of cardiovascular disease (CVD) an immunoassay to measure aggrecan fragments in said sample, and association of an elevation above a normal level with the presence of CVD, is conducted by contacting aggrecan fragments in said sample with an first antibody reactive with an N-terminal first epitope formed by cleavage of aggrecan by a proteinase and with a second antibody reactive with a second aggrecan epitope which is present in aggrecan at a location in the C-terminal direction from the location of said N-terminal epitope, and measuring the extent of simultaneous binding of both antibodies.

### CROSS REFERENCE TO RELATED APPLICATIONS

This application claims priority to U.S. provisional application Ser. No. 61/268,224, filed Jun. 9, 2009, the entire disclosure of which is incorporated herein by this reference.

### SEQUENCE LISTING

A listing of amino acid sequences appearing herein is filed herewith.

### BACKGROUND OF THE INVENTION

The present invention relates to assays for detection of biochemical markers valuable for diagnostic purposes in cardiovascular disease and prognosis of disease development, including biochemical markers indicative of the risk of cardiovascular events resulting from atherosclerotic development and plaque instability. In particular, the present invention relates to the detection of aggrecan and its fragments.

Worldwide, cardiovascular disease (CVD) is the leading cause of morbidity and mortality. At present, there are no effective and non-invasive diagnostic methods that allow for diagnosis and classification of patients into different risk-groups and for the diagnosis of low risk patients. Diagnostic and prognostic tools are composed mainly of multivariate analysis of simple markers, such as age, smoking and various lipid and lipoprotein concentrations.

CVD covers several clinical syndromes, primarily, angina pectoris, myocardial infarction (coronary thrombosis) and stroke. All of these syndromes are usually the sequelae of complicated atherosclerosis.

Atherosclerosis begins with intimal thickening in childhood and progresses to fatty streaks in the intima of arteries—these lesions are characterized as type I and II, respectively. Fatty streaks are the earliest macroscopically visible lesions in the development of atherosclerosis and occur among almost all human beings of all races and societies. In the non pathogenic state, endothelial cells (EC) resist adhesive interactions with leukocytes. However, the actions of proinflammatory cytokines and accumulated oxidized lipoprotein in the arterial wall during atherogenesis, initiate expression of adhesion molecules, such as intercellular adhesion molecules (ICAM)-1 and vascular cell adhesion molecules (VCAM)-1, on the surface of aortic ECs. This allows for capturing and transmigration of leukocytes through the endothelial surface, into the intimal part of the vessel wall. The development of plaques involves an increasing number of smooth muscle cells (SMC) that undergo displacement and apoptosis, which results in increased matrix turnover. The impaired collagen synthesis can result in a weakened fibrous cap and an atherosclerotic plaque that is more prone to rupture; however, most investigators believe that the actions of a proteolytic enzymes such as matrix metallo-proteases (MMPs) and other proteases importantly contribute to the risk of plaque rupture (Clarkson and Kaplan 509-28).

Plaques are divisible into two different types: ‘vulnerable’ and ‘stabilized’ plaques. However, for detailed histological analyses and molecular understanding, a more detailed classification is often used. There are three major stages in development of plaque: initiation, fatty streaks and the complex/advanced plaque (Stary H. C.).

Atherosclerotic plaques develop within the intima of arteries, and may be classified depending on their composition and structure. This classification divides lesions into eight types (Stary H. C.):
I. Macrophages loaded with and enlarged by lipid droplets (macrophage foam cells) are increased in the intima.
II. Macrophage foam cells accumulate in the deep part of the proteoglycan layer along with lipid droplets within the intimal SMC. The layers of foam cells are visible as fatty streaks. In type II lesions monocytes penetrate the endothelial lining by monocyte chemo attractant proteins (mainly MCP-1), which are over expressed in human atheroma. The early types of lesion (type I and II) can start in infancy and do not necessarily lead to plaque rupture. Furthermore, the development of atherosclerosis may end after the formation of type III lesion, and the formation of plaque is not predictable (Stary H. C.).
III. The type III lesion is determined as the intermediate lesion between the fatty streaks (type II) and the atheroma (type IV). These lesions contain pools of extracellular lipid and thereby expand the spaces between the normally closely adjoining SMCs of the deep musculo-elastic layer of the intima. The pools of material may replace proteoglycans and collagen fibres that normally reside here, but this occurs with little impact at this stage of atherogenesis.
IV. The atheroma is the first clinical sign of atherosclerosis. Displacement of SMCs in the intima of arteries by accumulating extracellular pools of lipids and disruption of the intimal architecture is a hallmark of a type IV lesion. The formation of the lipid cores is the end result of this SMC displacement. Formation of a lipid core accounts for the increased wall thickening. The lipid core is a large and well delineated region of the deep intima where the normal structural elements of this part of the arterial wall have been replaced by densely packed foam cell remnants, free lipids droplets, cholesterol crystals and calcium particles. SMCs normally resident in this area are decreased or completely absent at this stage of atherosclerosis progression. Any remnant SMCs become widely dispersed and have developed elongated cell bodies and very often unusually thick basement membranes. At this stage, the development of a layer overlying the lipid core begins. This layer consists of collagen and proteoglycan-rich intercellular matrix, SMCs with and without lipid droplets, macrophages, and foam cells.
V. The response to type IV lesion is the formation of a reparative fibrous tissue matrix, forming a fibrous “cap”. Typically, these lesions will consist of layers of lipid cores and reparative tissue irregularly stacked on top of each other. Events such as hematoma and thrombus formation may additionally complicate these types of lesions. If not fatal, these lesion complications are integrated into the lesion and overgrown by a thin layer of reparative matrix tissue, consisting of collagens and proteoglycans. The content of extracellular matrix proteins collagen and proteoglycans increases in the atherosclerotic plaque during formation of the cap.
VI. The defects of the endothelium such as fissures, erosions, ulcerations, hematoma, thrombus, haemorrhage can if combined lead to more complicated lesion type designated type VI lesion.
VII. The lesion is often referred to as calcified lesion, where more than 50% of the lesion consists of mineral. In addition to calcifications, these lesions contain abundance of reparative fibrous connective tissue. When the SMCs trapped in this undergo apoptosis and disintegrate; their mineralized organelles become a part of the calcification.
VIII. The fibrotic lesion follows the calcific lesion. The fibrotic lesion may consist entirely of collagen and no lipid. (Stary H. C.)

Cardiovascular events are often the result of plaque rupture, in which inflammation and the release of proteases weaken the shoulder regions of the fibrous cap and allow the fatty materials in the plaque to come into contact with the blood precipitating a mural thrombus (Clarkson and Kaplan). Thinning of the fibrous cap by increased protease activity in the combination with decreased matrix production, is considered a hallmark of plaque instability increasing the risk of rupture. Vulnerability of plaques and their risk of rupture is an area of clinical interest. Definition of a vulnerable plaque (VP) is not standardized, but there is a general agreement stating existence of three histological hallmarks compared to stable plaque:

1) A larger lipid core (>40 percent of total lesion).

2) A thinner fibrous cap (65-150 micrometers).

3) Large amount of acute inflammatory cells.

Major criteria for defining VP include: active inflammation (presence of monocytes, macrophages and T cells), thin cap with large lipid core, endothelial denudation with superficial platelet aggregation, fissured plaque, and >90% stenosis of the artery. Other minor criteria include: superficial calcified nodule, intraplaque haemorrhage, endothelial dysfunction, and outward remodelling (Shin, Edelberg, and Hong).

Plaque complications, instability and rupture may be inhibited by medical treatment and/or lifestyle modification. In some cases, however, more invasive methods may be needed, i.e. angioplasty or bypass surgery.

Presently, diagnostic tools are based on either static image analyses still under development or low-technology methods such as systolic and diastolic blood pressure levels related to the risk of CVD. The field has devoted much attention to the development of multivariate analysis that may better identify patients at high risk. One such model is the SCORE-model (Systematic Coronary Risk Evaluation model). In 1994, with a revision in 2003, The European Atherosclerosis Society, The European Society of Cardiology and The European Society of Hypertension issued a set of recommendations regarding prevention of coronary heart diseases. This guideline is based on several assessment techniques, which have been developed to assess the risk of CVD in asymptomatic subjects, i.e. identification of asymptomatic high-risk patients. The SCORE-model integrates gender, age, smoking, systolic blood pressure and either total cholesterol or the cholesterol/HDL ratio as risk factors (Graham et al.).

In order to make a more detailed diagnosis, the SCORE model is not sufficient and imaging techniques are used. Imaging methods are therefore used mostly on patients in the high-risk group or during research.

A range of different biochemical markers have been suggested as markers of cardiovascular events. Wang et al (2006) have measured 10 different biochemical markers in 3200 patients participating in the Framingham study, described in Table 1. The conclusion was that the measurement of 10 biochemical markers only contributes moderately to diagnosis over and above standard risk factors. Of the 10 biochemical markers, B-type natriuretic peptide level, C-reactive protein level and the urinary albumin-to-creatinine ratio showed the best correlation between marker and death/cardiovascular events (Wang et al.).

### Proteoglycans as Matrix Components

Proteoglycans (PG) are polysaccharide-protein macromolecules localized predominately in the intercellular matrix of vessel wall (Salisbury and Wagner 1981). PGs are macromolecules characterized by the presence of one, or more, long un-branched and highly polyanionic sugar side chains called GAGs, covalently attached to a core protein through a link region. The repeating unit of the GAG consists of an amino sugar, either N-acetyl-glucosamine (GlcNAc) or N-acetyl-galactosamine (GaINAc), and a hexuronic acid, either glucouronic acid (GlcA) or iduronic acid (IdoA). One or both of the sugars in the repeating unit contain one or more sulfate groups (Rodriguez-Lee 2007). In addition to the GAG chains, most core proteins carry N- and/or O-linked oligosaccharides.

### Classification and Nomenclature of PGs

PGs are a very heterogeneous group of macromolecules. A single type of core protein can vary in the number and type of attached GAG chains. The length of the chains and the arrangement of the sulfated residues along the chains vary also.

Four main classes of GAGs are distinguished according to the structure of the repeating disaccharide unit: chondroitin sulfate (CS) and dermatan sulfate (DS), heparin sulfate (HS) and heparin, hyaluronan, and keratin sulfate (KS).

Chondroitin/dermatan sulfate PGs (versican, aggrecan, neurocan, and brevican) belong to the family of hyaluronan-binding proteoglycans. This gene family is collectively termed hyalectans. Proteoglycans (PGs) are macromolecules distributed almost everywhere in the human body. The structure and size of PGs vary extremely. The basic structure of all PGs includes a core protein and at least one, but often many carbohydrate chains-glycosaminoglycans (GAGs). PGs can be found intracellularly, on the surface of cells, and in the extracellular matrix. Each family member has a characteristic distribution, with aggrecan prominent in cartilage, neurocan and brevican prominent in the central nervous system, and versican present in a variety of soft tissues, including arterial walls.

Aggrecan is heavily glycosylated and comprises more than 2000 amino-acid residues. Aggrecan is structurally organized in three distinct domains: G1, G2 and G3 (FIG. 1). Interspaced between the G2 and G3 domain, and to a lesser extent between the G1 and G2 domains are long stretches of heavily glycosylated regions, containing the negatively charged chondroitin sulphate and keratan sulphate oligosaccharide structures (Fosang 1995). The amino acid sequence of human aggrecan is given in SEQ ID NO 1.

### Protease Profiles

Proteases hydrolyse peptide bonds and are responsible for the degradation of extracellular matrix proteins such as collagen, proteoglycans and elastin in atheroma, see Table 1. In atherosclerotic plaques three main types are found: metallo-proteinases (i.e. MMPs), serine proteases and cysteine proteases (i.e. cathepsins). Cathepsins and MMPs are responsible for degradation of all extracellular matrix proteins. As matrix is essential for plaque stability, its removal from the fibrous cap by proteases may invoke plaque rupture (Stary H. C.).

In Table 1 a variety of proteases found in atherosclerotic plaque are listed.

TABLE 1
Proteases detected in atherosclerotic plaques.

| Protease | Degradation substrates |
|---|---|
| Cathepsin K | Proteoglycans, elastin, collagen |
| Cathepsin S | Proteoglycans, elastin, collagen |
| Cathepsin L | Proteoglycans, Collagen type I |
| Cathepsin B | Proteoglycans |
| MMP-1 | Collagen type I, II and III |
| MMP-2 | Proteoglycans, elastin |
| MMP-3 | Proteoglycans, collagen type III, |
| | elastin |
| MMP-8 | Proteoglycans, collagen type I, II |
| | and III |
| MMP-9 | Elastin, collagen type I and III |
| MMP-13 | Proteoglycans, collagen type I, II |
| | and III |
| MMP-18 | Collagen type I |

The main source of MMP expression in the plaque is suspected to be related to macrophage and SMC activity. Macrophages in plaques contain abundant MMP-1, -8, -9, and -13 and co-localize with sites of collagen and proteoglycan degradation in situ (Kunz J.). Furthermore, own data suggest localization of MMP-8 and Cathepsin K in atherosclerotic plaques.

### Matrix Metalloproteinases (MMP)

MMP is a large group of endopeptidases, capable of degrading most components of the ECM. Presently, more than 25 MMPs have been identified. Metallo-proteinases are characterized by an active site containing a metal atom, typically zinc, and are secreted as zymogens. Specific tissue inhibitors, TIMPs, regulate the activity of MMPs. A great variety of MMPs are found in the atherosclerotic plaques. They are most often located in macrophages bordering the fibrous cap, within plaque shoulders in SMC and macrophages and are rarely identified within the fibrous cap (Kunz J.).

MMPs are classified in different groups according to their substrate specificity: Collagenases, which degrade fibrillar collagen, like collagen type I, II, III and V but also proteoglycans; Gelatinases, which degrade proteoglycans, collagen type IV, V, VII and elastin; Stromelysin that is active against proteoglycans and elastin (Rouis M). These three subgroups are of particular interest with regards to matrix remodelling in atherosclerotic plaques.

### Gelatinases

Insoluble elastin is digested by MMP-2 and -9, both belonging to the gelatinase-family of MMPs. MMP-9 has an important role affecting the size and composition of atherosclerotic plaque. In unstable human atherosclerotic plaques and in vulnerable regions of plaques, greater expression and concentration of MMP-9 have been observed. Moreover, MMP-9 is found intracellularly (indicating active synthesis) in coronary plaques more often in patients with unstable angina compared with those with stable angina. Blood MMP-9 level increases in association with coronary atherosclerosis and predicts adverse cardiovascular events (Sundstrom and Vasan). A recent study by Kuzuya et al (2006) indicates that MMP-2 is responsible for accumulation of SMC in the fibrous cap and thereby inducing plaque instability.

### Stromelysin

MMP-3 belongs to the stromelysin proteases and is capable of degrading both elastin and proteoglycans. A study by Yamada et al (2002) indicates that MMP-3 may prove to be a reliable mean of predicting the genetic risk of myocardial infarction in women. Elevations of stromelysin occur in osteoarthritis, rheumatoid arthritis, atherosclerotic lesions, gout, inflammatory bowel disease (IBD), idiopathic pulmonary fibrosis (IPF), certain cancers, joint injuries, and numerous inflammatory diseases.

### Collagenases

MMP-1, -8 and -13 have all been identified in atherosclerotic plaques where they degrade proteoglycans and collagen types I and III.

MMP-1, -8 and -13 are collagenases, which cleave collagen into two fragments that are further degraded by MMP-2, -3 or -9.

MMP-8 is expressed by neutrophils, not commonly found in human atheroma but has been identified in atherosclerotic plaques. MMP-8 may be partly responsible for degradation of the fibrous cap as MMP-8 has a preference for collagen type I (Herman et al), having a three fold greater activity in degradation of collagen I than MMP-1 and 13. This is supported by Turu et al (2006), in this study the content of MMP-8 in the plasma are significantly higher for patients with vulnerable plaques, than patients with stable plaques.

MMP-13 has been reported to cleave SLRPS, with high specificity for biglycan. Degradation of biglycan by MMP-13 at a specific cleavage site ( . . . G₁₇₇/V₁₇₈) has previously been demonstrated by Monfort et al. (2005) and proposed to play a important role in early detection of cartilage degradation in osteoarthritis.)

### Cathepsins

Human cysteine cathepsins consist of 11 members, including cathepsins B, K, L, and S, and are predominantly expressed within the endosomal/lysosomal compartments of cells. Cathepsins are capable of catalysing the hydrolytic breakdown of proteoglycans, collagen and elastin.

In abdominal aortic aneurysm (AAA) high levels of cathepsins S, K, and L were found compared to normal aorta. Normal human vascular SMC contain no detectable cathepsin K by immunostaining, but cells within atherosclerotic plaques are clearly positive. Cathepsin K is localized in rupture-prone areas such as the fibrous cap, plaque shoulders and at the actual site of plaque ruptures (Chapman et al). Cathepsin S is found to co-localize with regions of increased elastin breakdown in atherosclerotic plaques, and reduced atherosclerosis is observed in cathepsin S- and K-deficient mice (Liu et al).

Both cathepsin L and K degrade several proteoglycans and collagen type I and II, cathepsin K degrades within covalently cross-linked triple helices, while cathepsin L cleaves only in the nonhelical telopeptide regions. Cathepsin K is localized in the fibrous cap and plaque shoulder. Cathepsin K expression in normal arteries is very low. Early human atherosclerotic lesions showed cathepsin K expression in the intimal and medial SMCs. In advanced atherosclerotic plaques, cathepsin K was localized mainly in macrophages and SMCs of the fibrous cap (Lutgens et al). Cathepsin K protein levels were increased in atherosclerotic lesions when compared with normal arteries, whereas cathepsin K mRNA levels were similar in both atherosclerotic and normal arteries. Furthermore, it was shown that cathepsin K mRNA and protein levels were highest in advanced but stable human atherosclerotic plaques compared with early atherosclerotic lesions and lesions containing thrombus (Chapman et al).

Cathepsin S is only sparsely expressed in intimal and medial SMCs in early human atherosclerotic lesion and fatty streaks. In advanced human atherosclerotic plaques cathepsin S was localized in macrophages and SMCs of the fibrous cap. EC lining the lumen of the vessel itself and the plaque microvessels also expressed cathepsin S. Furthermore, cathepsin S mRNA and protein levels were increased in human atheroma compared with normal arteries (Lutgens et al). Cathepsin S can degrade proteoglycans, elastin and collagen (Liu et al).

Presently, the determination of CVD risk is occurring at a late stage in atherosclerosis progression; a point in which there is a significant risk of fibrous plaque rupture. There is a need for diagnostic or prognostic assays that will provide information regarding atherosclerosis or CVD risk at both earlier stage and late stages. The findings of Katsuda et al (1992) suggest that there are enzymatic mechanisms for removal of collagens from advanced lesions, suggesting indeed a major role of neo-epitopes in arteriosclerosis.

### Assays for Detection of Aggrecan and its Fragments

Several immunoassays for detection of aggrecan and its fragments have been described, however, none of these assays have been demonstrated to be useful for assessment of CVD. Most such assays are concerned with cartilage degradation, but U.S. Pat. No. 5,387,504 describes the neo-epitope VDIPEN released by the action of stromelysin at the site N_{34I}-F₃₄₂ of aggrecan and an RIA assay employing a single monoclonal antibody specific for this epitope. It is alleged that such an assay might be useful in the diagnosis of various diseases including atherosclerotic lesions and also that aggrecan fragments arising from cartilage will be detectable this way in synovial fluid, blood, urine or other biological fluids as a measure of stomelysin activity or the effect of a stromelysin inhibitor. However, there is no data presented to support these suggestions. Nor are we aware of any being subsequently published.

RIA immunoassays for the VIDIPEN sequence have been available for many years now and have never been shown to be useful for measuring VIDIPEN containing peptides in blood or other similar body fluids as a diagnostic for any indication.

U.S. Pat. No. 5,387,504 indicates that the N-terminal sequence generated by cleavage at the N_{34I}-F₃₄₂ site may remain attached to cartilage, or may be released into synovial fluid. It suggests that assays to measure such fragments may be useful in characterising stomolysin inhibitors by monitoring release of such fragments into fluids including blood. The production of rabbit polyclonal antiserum useful to quantify large fragments from cartilage bearing the N-terminal sequence FFVG . . . is disclosed. General protocols for obtaining monoclonal antibodies are described also.

Numerous proteolytic clevage-sites have been described for aggrecan (Fosang et al ., 2000; Caterson et al., 2000), and a predominant site for the metalloproteinases (MMPs) is located in the intra-globular domain (IGD) between amino acid N³⁴¹ and F³⁴² (Fosang et al. 1996). A monoclonal antibody, i.e. AF28 (ATCC HB11671), that specifically binds the polypeptide neo-epitope containing the N-terminal sequence ³⁴²FFGVG . . . , has previously been developed (Fosang et al. 1995). The AF28 antibody has been used in competition ELISA for detection of aggrecan fragments in synovial fluid and human serum (Fosang et al. 1995).

Sumer et al. (2006) discloses two immunoassays, one of them detecting aggrecan fragments carrying both the neo-epitope ³⁴²FFGVG and the globular domain G2. A capture antibody (AF28) binding to the neo-epitope ³⁴²FFGVG was biotinylated and incubated on streptavidin-coated microtitre plates. Another antibody (F78) binding the G2 domain of aggrecan (but also having the ability to bind the G1 domain) was labelled with horseradish peroxidase, and used as detector antibody. Levels of the ³⁴²FFGVG-G2 fragments are slightly elevated in patients with rheumatoid arthritis, however, it did not reach statistical significance above a control population. Measurement of aggrecan fragments in patients with CVD symptoms was not reported.

Pratta et al. (2006) discloses a sandwich assay for detection of aggrecan fragments employing a monoclonal antibody to keratan sulfate as capture antibody and another monoclonal antibody to the neo-epitope ARGSVIL as detector antibody. This assay detects aggrecan fragments in human synovial fluid, however, measurements in human serum or plasma have not been reported and relevance to CVD is not suggested.

Karsdal et al. (2008) describes an immunoassay similar to that of Sumer above, except that the capture antibody was substituted with a monoclonal antibody (BC-3) binding to the neo-epitope ³⁷⁴ARGSVIL. The test was not used for measurements in human serum and no reference was made to relevance to CVD diagnosis.

More generally the use of monospecific antibodies specific for fragments of aggrecan, generated by specific stromelysin cleavage have been described. Until now, the clinical value of assays specific for these aggrecan ‘neo-epitopes’ has not been established and neither has it previously been established whether these fragments are released into circulation in significant amounts and how they are catabolised.

The CS 846 test uses antibodies recognising the chondroitin sulfate sidechain bound to amino acid 846 between the G2 and the G3 domain of the aggrecan molecule (IBEX Pharmaceuticals Inc.) (Glant et al., 1986; Rizkalla et al., 1992; Månsson et al., 1995). It is reported to be an assay for elvated fetal like aggrecan synthesis. The FA-846 sandwich immunoassay, which is an adaptation of the CS 846 test for the quantification of fetal aggrecan, has also been described.

Other tests for aggrecan have been developed, e.g. “Aggrecan Proteoglycan” (Biosource, US). However, the specificity of the antibodies remains to be determined. Other aggrecan assays target the glycosaminoglycan region of aggrecan, i.e. between the G2 and the G3 domain (Kongtawelert and Ghosh 1990).

Møller et al. have developed a competition ELISA for the core protein part of aggrecan, though not specifying the binding region of the antibody (Møller et al. 1994).

The antibody, l-C-6 has been developed which binds to both the G1 and un-masked G2 domains (Fosang and Hardingham, 1991). The G2 domain masking keratan sulphate side chains had to be removed using keratanase for reactivity with l-C-6 with the G2 domain. Accordingly, the l-C-6 antibody is not suitable for use in assays for aggrecan or aggrecan fragments in body fluids or body tissues.

Aggrecan is referred to in a number of patent publications. Several of these refer to measurement of aggrecan or certain characteristic fragments of the protein with a diagnostic purpose to assess cartilage catabolism.

U.S. Pat. No. 4,704,356 discloses that abnormal levels of keratan sulfate (KS) in the peripheral blood are indicative of abnormalities of cartilage or cartilage-like tissues. Elevated levels of KS in the peripheral blood are described as being indicative of osteoarthritis. Interestingly absence of KS as well as very elevated levels of KS in the peripheral blood were found to be indicative of muscular dystrophy and related disorders. The technique used for quantification of KS in the peripheral blood was an immunoassay using a monoclonal antibody.

U.S. Pat. No. 5,935,796 describes other diagnostic methods and compositions relating to the proteoglycan proteins of cartilage breakdown. Methods are described for early diagnosis, monitoring and treatment of osteoarthritis using monoclonal antibodies which specifically recognize antigenic determinants on atypical chondroitin sulfate (CS)/dermatan sulfate glycosaminoglycan chains in body tissues and fluids, that originate from articular cartilage aggrecan.

U.S. Pat. No. 4,778,768 describes methods for monitoring the progressive destruction of articular cartilage in joints, and more specifically for determining changes occurring in articular cartilage. The method involves (a) quantifying proteoglycan monomer and/or antigenic fragments thereof in a synovial fluid sample and (b) correlating the values thus obtained with progressive destructions in the articular cartilage appertaining to that sample fluid. The proteoglycan fragments were measured by an immunoassay employing an antibody specific to proteoglycan monomers. The assay described in this patent appears to be identical with the polyclonal HABr ELISA described above.

U.S. Pat. No. 5,948,692 describes an assay, which uses a size separation method for dividing glycans having avidity for hyaluronic acid (HA) from proteoglycans not having such avidity. The assay measures the HA binding proteoglycans, such as aggrecan. This is said to enable the biochemical diagnosis of joint diseases in the field of orthopedics as well as RA, OA and other joint diseases. The method can it is said be utilized also for discriminating normal joints from pathologic joints, for providing a prognostic measure of disease progression and for monitoring the effects of therapeutic interventions.

U.S. Pat. No. 5,427,954 describes the use of an immunoassay for measurement of aggrecan containing a neo-epitope ARGSVI. This is one of a number of disclosures describing the diagnostic utility of neo-epitopes generated by specific proteolysis of aggrecan mediated by proteases involved in the pathological processes of joint diseases.

U.S. Pat. No. 5,935,796 relates to methods and compositions for early diagnosis, monitoring and treatment of cartilage degenerative conditions, using an antibody which recognizes a peptide comprising the sequence FFGVG generated by cleavage of cartilage aggrecan at the site N₃₄₁-F₃₄₂. This epitope is the ‘other end’ of the VDIPEN epitope released by the action of stromelysin on aggrecan. It is suggested to provide a sandwich assay to improve the sensitivity of detection of FFGVG fragments of aggrecan, more specifically a sandwich assay combining AF-28 with an anti-keratan sulphate antibody such as 5-D-4.

U.S. Pat. No. 5,185,245 describes an immunoassay for detection of proteoglycans in synovial fluid and methods of monitoring treatment of diseases characterised by breakdown of proteoglycans. A test sample of synovial fluid is quantified by an immunoassay employing antibodies specifically recognizing proteoglycan, where the antibodies are immobilized on a solid support. Bound proteoglycan is then contacted with a second specific antibody, which is labeled with a detection reagent (i.e. peroxidase). Both antibodies have affinity to the glycosaminoglycan (GAG/CS) moieties on the proteoglycan.

U.S. Pat. No. 5,354,662 and U.S. Pat. No. 5,217,903 describe generally the measurement of ‘tissue breakdown products’ in body fluids based on quantification of a connective tissue or muscle tissue breakdown product in a body fluid from an animal by using a standard comprising the breakdown product having a radioactive label. The standard should have a known specific activity and thus combining the standard and a sample of the body fluid, the specific radioactivity measured in a RIA/IRMA type assay can be used as a measure of the quantity of the breakdown product in the sample. Also described are methods for assessing, in a body fluid from an animal, the condition of a selected connective tissue or a muscle tissue in an animal, and for assessing a disease process that includes destruction of a specified connective tissue component or muscle tissue, and for assessing the efficacy of a therapy for treatment of such a disease process, include the steps of the method for determining the quantity of a tissue breakdown product.

WO2007/045661 discloses assays for detection of aggrecan fragments using antibodies recognising epitopes located on the G2 domain of aggrecan, which are able to bind to aggrecan without prior removal of keratan sulfate by keratanase and using neo-epitope recognising antibodies. The patent application does not disclose the use of detecting aggrecan fragments for assessment of CVD.

### BRIEF SUMMARY OF THE INVENTION

It has now surprisingly been discovered that the concentration of certain aggrecan fragments is markedly elevated in certain clinical conditions associated with CVD, including coronary heart disease.

The present invention provides a method of diagnosis of cardiovascular disease (CVD) comprising a method of diagnosis of cardiovascular disease (CVD) comprising obtaining a patient biofluid sample, conducting an immunoassay to measure aggrecan fragments in said sample, and associating an elevation of said measure in said patient above a normal level with the presence of CVD, wherein said immunoassay is conducted by a method comprising: contacting aggrecan fragments in said sample with an first immunological binding partner reactive with an N-terminal first epitope formed by cleavage of aggrecan by a proteinase and with a second immunological binding partner reactive with a second aggrecan epitope which is present in aggrecan at a location in the C-terminal direction from the location of said N-termainal epitope, and measuring the extent of simultaneous binding of aggrecan fragments to both said first and said second immunological binding partners to measure therein aggrecan fragments comprising both of said first and said second epitopes.

The result of said assay may produce an index indicative of the degree of risk in a particular patient of rupture of an atherosclerotic plaque or of the vulnerable status of the atherosclerotic plaques of a patient.

Patients having a value for said index above a threshold level may be recommended for further investigation by plaque imaging methods (including those discussed above) or for the prescribing of medication for treatment of atherosclerosis or for surgical treatment of atherosclerosis, and such follow up investigations or treatment may form part of the method of the invention. Alternatively they may be selected for inclusion in a clinical trial of a therapeutic entity.

The method according to the invention may include comparing the measured amount of aggrecan fragments with a previously measured range of comparable values obtained for samples from a first group of patients having no cardiovascular disease and from a second group of patients previously diagnosed as having cardiovascular disease.

The method of the invention may include conducting the defined assay on samples derived from a first comparator patient group having no cardiovascular disease and conducting the defined assay on samples derived form a second comparator patient group having known cardiovascular disease, to obtain said range of comparable values.

The neoepitope is preferably located between the G1 and G2 domains.

### DETAILED DESCRIPTION OF PREFERRED EMBODIMENTS

### Aggrecan Assays

Aggrecan fragments may be fragments generated by any of the proteases present in atherosclerotic arteries.

Several candidate proteases may be responsible for the digestion of aggrecan in the plaque, as the literature reports many different proteases in the atherosclerotic plaques. Most likely, this is the result of a large range of complicated processes eventually leading to plaque rupture. However, in our assessment, early phases may consist of a range of MMPs, whereas later stages may rely more on cathepsin degradation of the matrix, resulting in different neo-epitope profiles dependent on the stages of the disease. Also, aggrecanases are present in atherosclerotic plaques and could therefore generate aggrecan fragments.

List of protease sites in aggrecan (AG)

TABLE 2
Protease sites in the
interglobular domain of aggrecan.

| AG sequence | SEQ ID NO | Cleavage Enzyme |
|---|---|---|
| VDIPEN*FFGVGG | 2 | |
| NITEGE*ARGSVI | 3 | |
| ILTVKP*IFEVSP | 4 | |
| AFTSED*LVVQVT | 5 | |
| AFCFRG*ISAVPS | 6 | |

*indicates a site ofcleavage.

Accordingly, in a method of the invention, said aggrecan fragments preferably comprise an N-terminal neo-epitope formed by cleavage of aggrecan by a protease at a site marked by the sign * in any one of the above partial sequences thereof.

Preferably, said first immunological binding partner is not reactive with other PGs. Preferably, said immunological binding partner is not reactive with a said sequence listed above if prolonged past the respective N-terminal ends of generated fragments.

Suitable immunological binding partners may therefore be specifically reactive with any of the following sequences in Table 3 at the N terminal of a peptide:

TABLE 3
N-terminal sequences of protease generated
peptide fragments of aggrecan

| Aggrecan neo-epitope, N-term. | SEQ ID NO |
|---|---|
| *FFGVGGEEDI | 7 |
| *ARGSVILTVK | 8 |
| *IFEVSPSPLE | 9 |
| *LVVQVTAVPG | 10 |
| *ISAVPSPGEE | 11 |

Further cleavage sites defining neo-epitopes that may be assayed in a similar manner can be identified by exposing aggrecan to any of the enzymes described herein and isolating and sequencing peptides thereby produced.

In particular, aggrecan fragments carrying both one of the above mentioned neo-epitopes and a larger globular domain, e.g. globular domain 2, have been demonstrated to be particular useful for detecting abnormalities associated with CVD.

Therefore, in one embodiment of the invention, the assay uses two antibodies (or more generally, immunological binding partners) in a sandwich construction, one antibody detecting the neo-epitope and the other antibody binding to the globular domain of aggrecan.

Aggrecan specificity is not necessary however in the case of the second antibody, because it is adequately provided by the first antibody. Accordingly, the second antibody may be one which is specifically reactive with the aggrecan fragments containing the first epitope, but by virtue of being reactive with a structure which is not unique to aggrecan, for instance by binding keratan sulphate.

Assays for more than one of the peptides described above may be conducted separately and their results combined or more than one of the peptides described above may be measured together.

The result of an assay according to the invention may be combined with one or more other measured biomarkers to form a composite index of diagnostic or prognostic value.

The term ‘immunological binding partner’ as used herein includes polyclonal and monoclonal antibodies and also specific binding fragments of antibodies such as Fab or F(ab′)₂. Thus, said immunological binding partner may be a monoclonal antibody or a fragment of a monoclonal antibody having specific binding affinity.

Generally, all previously known sandwich immunoassay formats can be used in accordance with this invention including heterogeneous and homogeneous formats enzyme linked assays, radio-immune assays and the like.

A suitable method could be a sandwich assay using two different antibodies, preferable monoclonal antibodies. One antibody binding to the neo-epitope as described above, and the other antibody recognising one of the globular domains of aggrecan, i.e. globular domain 2 (G2) or 3 (G3). The neo-epitope binding antibody could be used for coating of microtitre plates, which are subsequently incubated with a sample suspected to contain aggrecan fragments derived from atherosclerotic plaques. Next, the wells of the plates are incubated with the second antibody binding to G2 or G3, and this antibody could be labelled for detection, e.g. with horseradish peroxidise or other suitable label.

In certain preferred methods, the sample is a patient derived sample, and the method further comprises comparing the determined level of said binding of said peptide fragments with values characteristic of (a) comparable healthy individuals and/or (b) a pathological atherosclerotic condition and optionally associating a higher level of the measured peptide (normally indicated by a higher level of binding) with a more severe degree of a said condition.

An aspect of the present invention relates to the development of monoclonal antibodies recognising neo-epitopes as described above. This can be achieved by immunising mice with synthetic peptides originating from the amino acid sequence of the protein molecule concerned (including the sequences listed above or sequences terminating therein), fusing the spleen-cells from selected mice to myeloma cells, and testing the monoclonal antibodies for binding to neo-epitopes on relevant synthetic peptides. Specificity for neo-epitopes can be ensured by requiring reactivity with a synthetic peptide and a lack of reactivity with either a C-prolongated form of the immunising peptide (for a C-terminal neo-epitope) or an N-terminal prolongated form of the immunising peptide (for an N-terminal neo-epitope). Antibodies for neo-epitopes may also be evaluated to establish a lack of binding capacity to native protein. Alternatively, specificity for a neo-epitope can be ensured by requiring the reactivity of the antibody to be negatively dependent on the presence of biotin or other functional groups covalently linked to one of the terminal amino acids.

The invention will make use of an immunological binding partner which is specifically immunoreactive with a neo-epitope formed by cleavage of aggrecan by a protease at an end-site in any one of the partial sequences set out above, and may be for instance a monoclonal antibody or a binding fragment thereof.

The invention may make use of a cell line producing a monoclonal antibody against an N-terminal neo-epitope formed by cleavage of an atherosclerotic plaque protein at the end-sites of sequences in any one of the partial sequences o set out above.

The invention may further make use of a peptide comprising an N-terminal neo-epitope formed by cleavage of aggrecan in any one of the partial sequences of these proteins set out above. Such a peptide may be conjugated as a hapten to a carrier for producing an immune response to said peptide, or immobilised to a solid surface or conjugated to a detectable marker for use in an immunoassay.

The invention may employ methods for the development of monoclonal antibodies recognising the globular domains of aggrecan as described above. This can be achieved by immunising mice with purified, intact aggrecan, fusing the spleen-cells from selected mice to myeloma cells, and testing the monoclonal antibodies for reactivity to intact aggrecan. Specificity for aggrecan can be ensured by demonstrating lack of reactivity to other proteoglycans, e.g. by showing that the binding of the monoclonal antibodies to aggrecan cannot be inhibited by coincubation with said other proteoglycans.

The invention may make use of an immunological binding partner which is specifically immunoreactive with one of the globular domains, i.e. G2 or G3, and may be for instance a monoclonal antibody or a binding fragment thereof.

The invention may make use of a cell line producing a monoclonal antibody against intact aggrecan.

The invention may further make use of an isolated nucleic acid molecule coding for a peptide comprising an N-terminal neo-epitope formed by cleavage of aggrecan in any one of the partial sequences set out above.

The invention may further make use of a vector comprising a nucleic acid sequence comprising an expression signal and a coding sequence which codes for the expression of a peptide comprising an N-terminal neo-epitope formed by cleavage of aggrecan in any one of the partial sequences set out above and further includes a host cell transformed with such a vector and expressing a said peptide.

The invention may be performed using kits, which may include (1) a microtitre plate coated with synthetic peptide; (2) a monoclonal antibody or antibody binding fragment of the invention reactive with said synthetic peptide characteristic of aggrecan; and (3) a labelled anti-mouse IgG immunoglobulin. Alternatively, such kits may include (1) a microtitre plate coated with purified native aggrecan fragments; (2) a monoclonal antibody recognising a neo-epitope on an aggrecan fragment, and reactive with said purified fragments; and (3) a labelled anti-mouse IgG immunoglobulin. Alternatively, such kits may include (1) a microtitre plate coated with streptavidin; (2) a synthetic peptide linked to biotin; (3) a monoclonal antibody recognising a neo-epitope on said aggrecan fragment and reactive with said synthetic peptide; and (4) a labelled anti-mouse IgG immunoglobulin. Yet another alternative could be kits including (1) a microtitre plate coated with streptavidin; (2) a synthetic peptide linked to biotin; (3) a monoclonal antibody recognising a neo-epitope on said aggrecan fragment (and reactive with said synthetic peptide) and conjugated to horseradish peroxidase. And yet another alternative, such kits may include; (1) a microtitre plate coated with a monoclonal antibody binding to a neo-epitope on aggrecan; (2) a monoclonal antibody recognising G2 or G3 on said aggrecan fragment and conjugated to horseradish peroxidise. Another alternative include kits containing; (1) a microtitre plate coated with streptavidin; (2) a biotin-labelled monoclonal antibody binding to a neo-epitope on said aggrecan fragment (3) a monoclonal antibody recognising G2 or G3 on said aggrecan fragment.

Thus, the invention may make use of an immunoassay kit comprising an immunological binding partner as described herein, and a competition agent which binds said immunological binding partner, and optionally one or more of a wash reagent, a buffer, a stopping reagent, an enzyme label, an enzyme label substrate, calibration standards, an anti-mouse antibody and instructions for conducting a said immunoassay.

Also, the invention may employ an immunoassay kit comprising two different immunological binding partners as described herein, and optionally one or more of a wash reagent, a buffer, a stopping reagent, an enzyme label substrate, and calibration standards, and instructions for conducting a said immunoassay.

The assays described herein are useful in the diagnosis of atherosclerotic disease in patients. In addition, the tests are useful for the assessment of disease progression, and the monitoring of response to therapy. The immunological binding partners of the invention may also be used in immunostaining to show the presence or location of cleavage products of any atherosclerotic plaque protein described herein.

### BRIEF DESCRIPTION OF THE DRAWINGS

The invention will be further explained and illustrated with reference to the accompanying drawings, in which:

FIG. 1 shows the structure of aggrecan.

FIGS. 2A, B, C and D show the detection of aggrecan fragments in serum samples from patients with congestive heart failure (CF) and coronary heart disease (CHD) using two assays according to the invention and two comparative methods. Control samples include serum samples from patients with congestive heart failure.

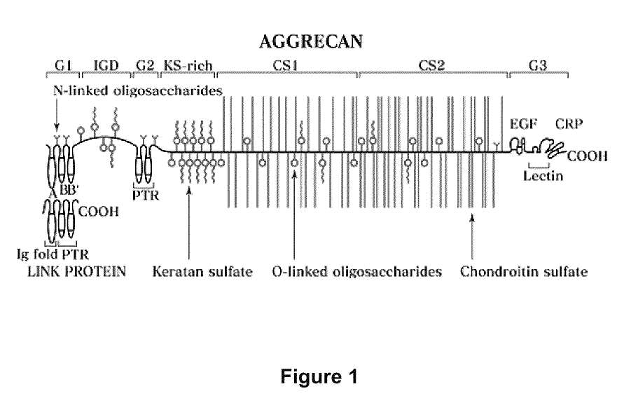

### EXAMPLE 1

### ³⁴²FFGVG-G2 Assay

An immune assay for detection of aggrecan fragments carrying both the neo-epitope ³⁴²FFGVG and the globular domain G2 was conducted as described by Sumer et al., 2006. Briefly, monoclonal antibody Af28 binding to the neo-epitope ³⁴²FFGVG (U.S. Pat. No. 5,935,796) was labelled with biotin and used for coating of streptavidin plates After incubating the plates for 1 hour, plates were washed 5 times with washing buffer (0.15 mol/1 NaCl, 0.05% (v/v) Tween 20). Subsequently 50 μl standards (MMP-13 digested purified bovine aggrecan (SIGMA) 47-3000 ng/ml or human serum prediluted 1:50 in PBS-BTB were added, and the plates were incubated for 1 hour, 300 RPM, 20° C. After the incubation period, the plates were washed 5 times as described previously, and 500 ng/ml horseradish Peroxidase (POD)-labelled F78 antibody diluted in PBS-BTB with blocking agent (Roche GmbH) was added. After incubating for 1 hour, 300 RPM, 20° C., the plates were washed 5 times, 100 μl of TMB substrate was added, and the plates were incubated for 15 minutes, 300 RPM, 20° C. in the dark before 150 μl 0.18 M H₂SO₄ was added. The absorbance was measured immediately after at 450 nm.

Human serum samples from patients with congestive heart failure (CF) and coronary heart disease (CHD) was measured in the assay. Control specimens originated from patients with various inflammatory diseases, including rheumatoid arthritis. Also, as control, the serum samples was evaluated for presence of aggrecan fragments containing G1 and/or G2 using antibody F78 as both capture and detector antibody.

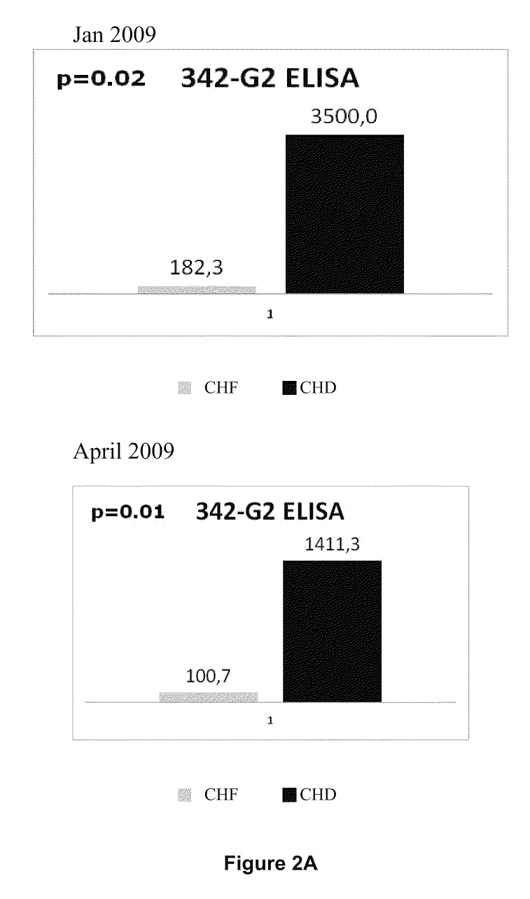

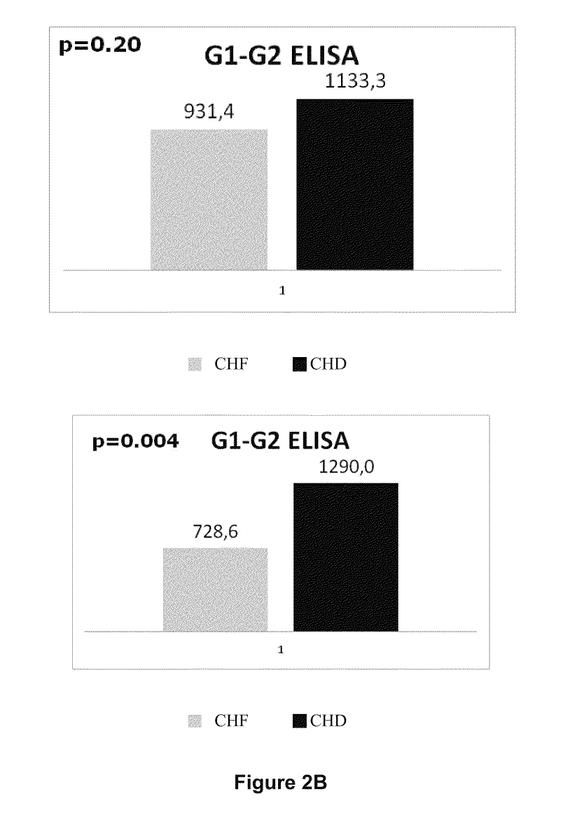

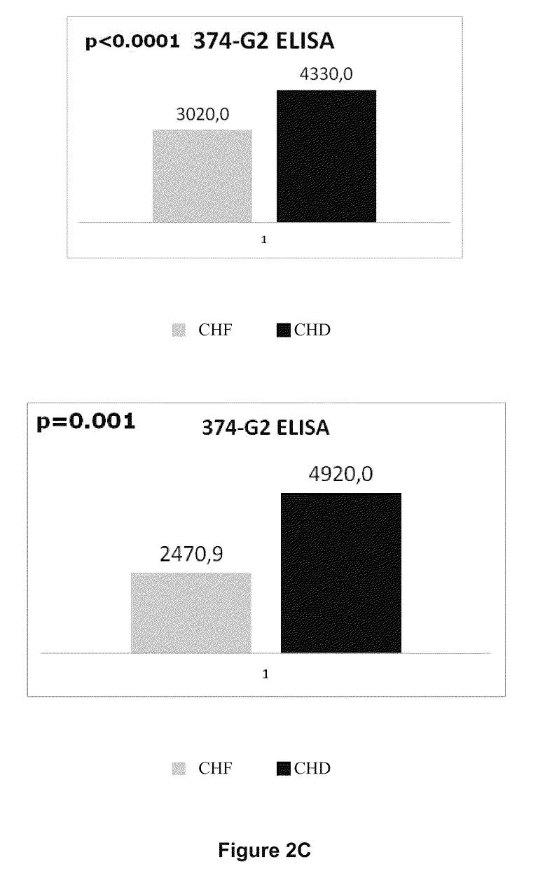

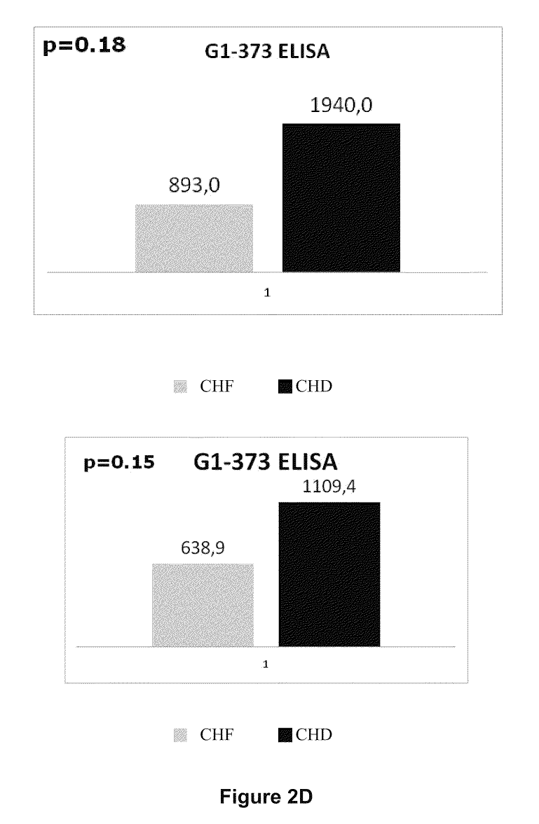

The test was carried out on two groups of samples, including in each group samples from patients with known CVD and patients with known CHD. In FIGS. 2A-D, results for the two groups are shown. A dramatic elevation in the serum concentration of 342FFGVG-G2 fragments was detected in patients with CHD (FIG. 2A). In contrast, only background levels of these fragments were detected in patients with CF and the control population.

### EXAMPLE 2

### 374ARGSV-G2 Sandwich ELISA

### Aggrecanase-Derived Aggrecan Fragments Carrying the 374ARGSV Neo-Epitope and the G2 Domain

Microtitre plates were coated with rabbit anti-mouse immunoglobulins diluted to 10 μg/ml and incubated overnight at 4° C., After washing, the wells were incubated with monoclonal antibody 6D6 diluted in to 500 ng/ml in PBS-BTE (PBS with 1% (w/v) BSA Tween 20 and EDTA, pH 7.4). MAb 6D6 binds specifically to the N-terminal neo-epitope ³⁷⁴ARGSV generated by proteolytic cleavage of aggrecan by aggrecanase. Following incubation for 1 hour at 20° C. with shaking and washing, the wells were incubated for another hour with 100 μL of human serum pre-diluted 1:20 in PBS-BT2 buffer (as PBS-BT1 but with 8 g/L of NaCl). Bovine aggrecan cleaved for 24 hours with ADAMTS-4 and diluted in PBS-BT2 buffer was used as calibrators. Bound antigen was detected by incubation with POD-labelled MAb F78 diluted to 2000 ng/ml in PBS-BT1 buffer (PBS with BSA, 0.8 g/L NaCl, and Tween 20) containing 10% Liquid II (Roche GmbH) for 1 hour at 20° C. with shaking. After washing the colour reaction was performed as described above.

Serum samples also used in Example 1 were evaluated in the 374ARGSV-G2 sandwich ELISA. Patients with coronary heart disease (CHD) had a concentration of circulating aggrecan fragments of 4330+194 ng/ml, whereas samples from patients with congestive heart failure (CHF) had 3020+372 ng/ml (p<0.0001), as shown in FIG. 2C.

### COMPARATIVE EXAMPLES

By way of comparison, ELISAs were conducted on the same serum samples to measure fragments comprising the G1 globular domain of aggrecan and a C-terminal neo-epitope which was . . . VIDIPEN in one case and . . . NITEGE in another. The results are seen in FIGS. 2B and C. No statistically significant difference is found in comparing CVD and CHF patients.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCE LIST

Caterson B, Flannery C R, Hughes C E, Little C B. Mechanisms involved in cartilage proteoglycan catabolism. Matrix Biol. 2000 August; 19(4):333-44. Review.
Chapman H A, Riese R J, Shi G P. Emerging roles for cysteine proteases in human biology. Annu. Rev. Physiol 1997; 59:63-88.
Clarkson T B, Kaplan J R. Stage of Reproductive Life, Atherosclerosis Progression and Estrogen Effects on Coronary Artery Atherosclerosis, In: Lobo R A, editor. Treatment of the Postmenopausal Woman: Basic and Clinical Aspects, 3 ed. San Diego: Elsevier; 2007. p. 509-28.
Fosang A J, Hardingham T E. 1-C-6 epitope in cartilage proteoglycan G2 domain is masked by keratan sulphate. Biochem J. 1991 Jan. 15;273(Pt 2):369-73.
Fosang A J, Last K, Maciewicz R A. Aggrecan is degraded by matrix metalloproteinases in human arthritis. Evidence that matrix metalloproteinase and aggrecanase activities can be independent. J Clin Invest. 1996 Nov. 15; 98(10):2292-9.
Fosang A J, Last K, Gardiner P, Jackson D C, Brown L. Development of a cleavage-site-specific monoclonal antibody for detecting metalloproteinase-derived aggrecan fragments: detection of fragments in human synovial fluids. Biochem J. 1995 Aug. 15; 310 (Pt 1):337-43.
Fosang A J, Last K, Gardiner P, Jackson D C, Brown L. Development of a cleavage-site-specific monoclonal antibody for detecting metalloproteinase-derived aggrecan fragments: detection of fragments in human synovial fluids. Biochem J. 1995 Aug. 15; 310 (Pt 1):337-43.

Glant T T, Mikecz K, Poole A R. Monoclonal antibodies to different protein-related epitopes of human articular cartilage proteoglycans. Biochem J. 1986 Feb. 15; 234(1):31-41.
Graham I, Atar D, Borch-Johnsen K, Boysen G, Burell G, Cifkova R et al. European guidelines on cardiovascular disease prevention in clinical practice: executive summary. Atherosclerosis 2007; 194:1-45.
Herman M P, Sukhova G K, Libby P, Gerdes N, Tang N, Horton D B et al. Expression of neutrophil collagenase (matrix metalloproteinase-8) in human atheroma: a novel collagenolytic pathway suggested by transcriptional profiling. Circulation 2001; 104:1899-904.
Karsdal M A, Madsen S H, Christiansen C, Henriksen K, Fosang A J, Sondergaard B C. Cartilage degradation is fully reversible in the presence of aggrecanase but not matrix metalloproteinase activity. Arthritis Res Ther. 2008; 10(3):R63.
Katsuda S, Okada Y, Minamoto T, Oda Y, Matsui Y, Nakanishi I. Collagens in human atherosclerosis. Immunohistochemical analysis using collagen type-specific antibodies. Arterioscler. Thromb. 1992; 12:494-502.
Kongtawelert P, Ghosh P. A new sandwich-ELISA method for the determination of keratan sulphate peptides in biological fluids employing a monoclonal antibody and labelled avidin biotin technique. Clin Chim Acta. 1990 Dec. 31; 195(1-2):17-26.
Kunz J. Matrix metalloproteinases and atherogenesis in dependence of age. Gerontology. 2007; 53:63-73.
Kuzuya M, Nakamura K, Sasaki T, Cheng X W, Itohara S, Iguchi A. Effect of MMP-2 deficiency on atherosclerotic lesion formation in apoE-deficient mice. Arterioscler. Thromb. Vasc. Bio12006; 26:1120-25.
Liu J, Sukhova G K, Sun J S, Xu W H, Libby P, Shi G P. Lysosomal cysteine proteases in atherosclerosis. Arterioscler. Thromb. Vasc. Biol 2004; 24:1359-66.
Lutgens, S. P., et al. “Cathepsin cysteine proteases in cardiovascular disease.” FASEB J. 21.12 (2007): 3029-41.
Månsson B, Carey D, Alini M, Ionescu M, Rosenberg L C, Poole A R, Heinegård D, Saxne T. Cartilage and bone metabolism in rheumatoid arthritis. Differences between rapid and slow progression of disease identified by serum markers of cartilage metabolism. J Clin Invest. 1995 March; 95(3):1071-7.
Møller H J, Larsen F S, Ingemann-Hansen T, Poulsen J H. ELISA for the core protein of the cartilage large aggregating proteoglycan, aggrecan: comparison with the concentrations of immunogenic keratan sulphate in synovial fluid, serum and urine. Clin Chim Acta. 1994 February; 225(1):43-55.
Monfort J, Nacher M, Montell E, Vila J, Verges J and Benito P, Chondroitin sulfate and hyaluronic acid (500-730 kda) inhibit stromelysin-1 synthesis in human osteoarthritic chondrocytes. Drugs Exp Clin Res. 2005; 31(2):71-6.
Pratta M A, Su J L, Leesnitzer M A, Struglics A, Larsson S, Lohmander L S, Kumar S. Development and characterization of a highly specific and sensitive sandwich ELISA for detection of aggrecanase-generated aggrecan fragments. Osteoarthritis Cartilage. 2006 Jul;14(7):702-13.
Rizkalla G, Reiner A, Bogoch E, Poole A R. Studies of the articular cartilage proteoglycan aggrecan in health and osteoarthritis. Evidence for molecular heterogeneity and extensive molecular changes in disease. J Clin Invest. 1992 December; 90(6):2268-77.
Rodriguez-Lee M, Bondjers G and Camejo G, Fatty acid-induced atherogenic changes in extracellular matrix proteoglycans. Curr Opin Lipidol. 2007 October; 18(5):546-53
Rouis M. Matrix metalloproteinases: a potential therapeutic target in atherosclerosis. Curr Drug Targets. Cardiovasc Haematol Disord. 2005; 5:541-48.
Rudel L L, Haines J, Sawyer J K, Shah R, Wilson M S, Carr T P. Hepatic origin of cholesteryl oleate in coronary artery atherosclerosis in African green monkeys. Enrichment by dietary monounsaturated fat. J Clin Invest 1997; 100:74-83.
Salisbury B G and Wagner, W DJ Biol Chem. 1981 Aug. 10; 256(15):8050-7, ‘Isolation and preliminary characterization of proteoglycans dissociatively extracted from human aorta’.
Shin, J., J. E. Edelberg, and M. K. Hong. “Vulnerable atherosclerotic plaque: clinical implications.” Curr. Vasc. Pharmacol. 1.2 (2003): 183-204.
Stary H C. Composition and classification of human atherosclerotic lesions. Virchows Arch A. Pathol Anat. Histopathol. 1992; 421:277-90.
Sumer E U, Sondergaard B C, Rousseau J C, Delmas P D, Fosang A J, Karsdal M A, Christiansen C, Qvist P. MMP and non-MMP-mediated release of aggrecan and its fragments from articular cartilage: a comparative study of three different aggrecan and glycosaminoglycan assays. Osteoarthritis Cartilage. 2007 February; 15(2):212-21.
Sundstrom J, Vasan R S. Circulating biomarkers of extracellular matrix remodeling and risk of atherosclerotic events. Curr Opin Lipidol. 2006; 17:45-53.
Turu M M, Krupinski J, Catena E, Rosell A, Montaner J, Rubio F et al. Intraplaque MMP-8 levels are increased in asymptomatic patients with carotid plaque progression on ultrasound. Atherosclerosis 2006; 187:161-69.
Wang T J, Gona P, Larson M G, Tofler G H, Levy D, Newton-Cheh C et al. Multiple biomarkers for the prediction of first major cardiovascular events and death. N Engl J Med 2006; 355:2631-39.
Yamada Y, Izawa H, Ichihara S, Takatsu F, Ishihara H, Hirayama H et al. Prediction of the risk of myocardial infarction from polymorphisms in candidate genes. N Engl J Med 2002; 347:1916-23.

The entire contents of all patents, published patent applications and other references cited herein are hereby expressly incorporated herein in their entireties by reference.

Those skilled in the art will recognize, or be able to ascertain using no more than routine experimentation, numerous equivalents to the specific procedures described herein. Such equivalents are considered to be within the scope of this invention and are covered by the following claims.

```
SEQUENCES REFERRED TO ABOVE:
SEQ ID NO 1
      1 MTTLLWVFVT LRVITAAVTV ETSDHDNSLS VSIPQPSPLR VLLGTSLTIP CYFIDPMHPV
    61 TTAPSTAPLA PRIKWSRVSK EKEVVLLVAT EGRVRVNSAY QDKVSLPNYP AIPSDATLEV
  121 QSLRSNDSGV YRCEVMHGIE DSEATLEVVV KGIVFHYRAI STRYTLDFDR AQRACLQNSA
  181 IIATPEQLQA AYEDGFHQCD AGWLADQTVR YPIHTPREGC YGDKDEFPGV RTYGIRDTNE
  241 TYDVYCFAEE MEGEVFYATS PEKFTFQEAA NECRRLGARL ATTGHVYLAW QAGMDMCSAG
  301 WLADRSVRYP ISKARPNCGG NLLGVRTVYV HANQTGYPDP SSRYDAICYT GEDFVDIPEN
  361 FFGVGGEEDI TVQTVTWPDM ELPLPRNITE GEARGSVILT VKPIFEVSPS PLEPEEPFTF
  421 APEIGATAFA EVENETGEAT RPWGFPTPGL GPATAFTSED LVVQVTAVPG QPHLPGGVVF
  481 HYRPGPTRYS LTFEEAQQAC PGTGAVIASP EQLQAAYEAG YEQCDAGWLR DQTVRYPIVS
  541 PRTPCVGDKD SSPGVRTYGV RPSTETYDVY CFVDRLEGEV FFATRLEQFT FQEALEFCES
  601 HNATATTGQL YAAWSRGLDK CYAGWLADGS LRYPIVTPRP ACGGDKPGVR TVYLYPNQTG
  661 LPDPLSRHHA FCFRGISAVP SPGEEEGGTP TSPSGVEEWI VTQVVPGVAA VPVEEETTAV
  721 PSGETTAILE FTTEPENQTE WEPAYTPVGT SPLPGILPTW PPTGAETEES TEGPSATEVP
  781 SASEEPSPSE VPFPSEEPSP SEEPFPSVRP FPSVELFPSE EPFPSKEPSP SEEPSASEEP
  841 YTPSPPEPSW TELPSSGEES GAPDVSGDFT GSGDVSGHLD FSGQLSGDRA SGLPSGDLDS
  901 SGLTSTVGSG LTVESGLPSG DEERIEWPST PTVGELPSGA EILEGSASGV GDLSGLPSGE
  961 VLETSASGVG DLSGLPSGEV LETTAPGVED ISGLPSGEVL ETTAPGVEDI SGLPSGEVLE
1021 TTAPGVEDIS GLPSGEVLET TAPGVEDISG LPSGEVLETT APGVEDISGL PSGEVLETAA
1081 PGVEDISGLP SGEVLETAAP GVEDISGLPS GEVLETAAPG VEDISGLPSG EVLETAAPGV
1141 EDISGLPSGE VLETAAPGVE DISGLPSGEV LETAAPGVED ISGLPSGEVL ETAAPGVEDI
1201 SGLPSGEVLE TAAPGVEDIS GLPSGEVLET AAPGVEDISG LPSGEVLETA APGVEDISGL
1261 PSGEVLETAA PGVEDISGLP SGEVLETTAP GVEEISGLPS GEVLETTAPG VDEISGLPSG
1321 EVLETTAPGV EEISGLPSGE VLETSTSAVG DLSGLPSGGE VLEISVSGVE DISGLPSGEV
1381 VETSASGIED VSELPSGEGL ETSASGVEDL SRLPSGEEVL EISASGFGDL SGVPSGGEGL
1441 ETSASEVGTD LSGLPSGREG LETSASGAED LSGLPSGKED LVGSASGDLD LGKLPSGTLG
1501 SGQAPETSGL PSGFSGEYSG VDLGSGPPSG LPDFSGLPSG FPTVSLVDST LVEVVTASTA
1561 SELEGRGTIG ISGAGEISGL PSSELDISGR ASGLPSGTEL SGQASGSPDV SGEIPGLFGV
1621 SGQPSGFPDT SGETSGVTEL SGLSSGQPGV SGEASGVLYG TSQPFGITDL SGETSGVPDL
1681 SGQPSGLPGF SGATSGVPDL VSGTTSGSGE SSGITFVDTS LVEVAPTTFK EEEGLGSVEL
1741 SGLPSGEADL SGKSGMVDVS GQFSGTVDSS GFTSQTPEFS GLPSGIAEVS GESSRAEIGS
1801 SLPSGAYYGS GTPSSFPTVS LVDRTLVESV TQAPTAQEAG EGPSGILELS GAHSGAPDMS
1861 GEHSGFLDLS GLQSGLIEPS GEPPGTPYFS GDFASTTNVS GESSVAMGTS GEASGLPEVT
1921 LITSEFVEGV TEPTISQELG QRPPVTHTPQ LFESSGKVST AGDISGATPV LPGSGVEVSS
1981 VPESSSETSA YPEAGFGASA APEASREDSG SPDLSETTSA FHEANLERSS GLGVSGSTLT
2041 FQEGEASAAP EVSGESTTTS DVGTEAPGLP SATPTASGDR TEISGDLSGH TSQLGVVIST
2101 SIPESEWTQQ TQRPAETHLE IESSSLLYSG EETHTVETAT SPTDASIPAS PEWKRESEST
2161 AADQEVCEEG WNKYQGHCYR HFPDRETWVD AERRCREQQS HLSSIVTPEE QEFVNNNAQD
2221 YQWIGLNDRT IEGDFRWSDG HPMQFENWRP NQPDNFFAAG EDCVVMIWHE KGEWNDVPCN
2281 YHLPFTCKKG TATTYKRRLQ KRSSRHPRRS RPSTAH
SEQ ID NO 2
VDIPENFFGVGG
SEQ ID NO 3
NITEGEARGSVI
SEQ ID NO 4
ILTVKPIFEVSP
SEQ ID NO 5
AFTSEDLVVQVT
SEQ ID NO 6
AFCFRGISAVPS
SEQ ID NO 7
FFGVGGEEDI
SEQ ID NO 8
ARGSVILTVK
SEQ ID NO 9
IFEVSPSPLE
SEQ ID NO 10
LVVQVTAVPG
SEQ ID NO 11
ISAVPSPGEE
```

1. A method of diagnosis of cardiovascular disease (CVD) comprising obtaining a patient biofluid sample, conducting an immunoassay to measure aggrecan fragments in said sample, and associating an elevation of said measure in said patient above a normal level with the presence of CVD, wherein said immunoassay is conducted by a method comprising:
contacting aggrecan fragments in said sample with an first immunological binding partner reactive with an N-terminal first epitope formed by cleavage of aggrecan by a proteinase and with a second immunological binding partner reactive with a second aggrecan epitope which is present in aggrecan at a location in the C-terminal direction from the location of said N-termainal epitope, and measuring the extent of simultaneous binding of aggrecan fragments to both said first and said second immunological binding partners to measure therein aggrecan fragments comprising both of said first and said second epitopes.

2. A method as claimed in claim 1, further comprising comparing the measured amount of aggrecan fragments with a previously measured range of comparable values obtained for samples from a first group of patients having no cardiovascular disease and from a second group of patients previously diagnosed as having cardiovascular disease.

3. A method as claimed in claim 1, wherein said first immunological binding partner has specific binding affinity for an N-terminal peptide sequence selected from the group consisting of:

| | | Cleavage |
|---|---|---|
| | | site |
| Amino acid sequence | SEQ ID NO | location |
| *FFGVGGEEDI . . . | 7 | 342 |
| *ARGSVILTVK . . . | 8 | 374 |
| *IFEVSPSPLE . . . | 9 | |
| *LVVQVTAVPG . . . | 10 | |
| *ISAVPSPGEE . . . | 11 | |

4. A method as claimed in claim 1, wherein said second immunological binding partner has specific binding affinity for the G2 globular domain of aggrecan.

5. A method as claimed in claim 4, wherein said second immunological binding partner has specific binding affinity for said globular domain of aggrecan even when said domain bears keratan sulphate chains.

6. A method as claimed in claim 1 or claim 2, wherein said second immunological binding partner has specific binding affinity for keratan sulphate.

7. A method as claimed in claim 1, wherein said immunoassay is conducted as a sandwich immunoassay.

8. A method as claimed in claim 1, wherein said sample is a blood sample or a blood derived sample.


SEQUENCE LISTING (18 sequences)

SEQ ID NO: 1 - protein, 2316 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGHVYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACPGTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAETEESTEGPSATEVPSASEEPSPSEVPFPSEEPSPSEEPFPSVRPFPSVELFPSEEPFPSKEPSPSEEPSASEEPYTPSPPEPSWTELPSSGEESGAPDVSGDFTGSGDVSGHLDFSGQLSGDRASGLPSGDLDSSGLTSTVGSGLTVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGVPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGVSGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAADQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTATTYKRRLQKRSSRHPRRSRPSTAH

SEQ ID NO: 2 - protein, 12 aa, Homo sapiens
VDIPENFFGVGG

SEQ ID NO: 3 - protein, 12 aa, Homo sapiens
NITEGEARGSVI

SEQ ID NO: 4 - protein, 12 aa, Homo sapiens
ILTVKPIFEVSP

SEQ ID NO: 5 - protein, 12 aa, Homo sapiens
AFTSEDLVVQVT

SEQ ID NO: 6 - protein, 12 aa, Homo sapiens
AFCFRGISAVPS

SEQ ID NO: 7 - protein, 10 aa, Homo sapiens
FFGVGGEEDI

SEQ ID NO: 8 - protein, 10 aa, Homo sapiens
ARGSVILTVK

SEQ ID NO: 9 - protein, 10 aa, Homo sapiens
IFEVSPSPLE

SEQ ID NO: 10 - protein, 10 aa, Homo sapiens
LVVQVTAVPG

SEQ ID NO: 11 - protein, 10 aa, Homo sapiens
ISAVPSPGEE

SEQ ID NO: 12 - protein, 6 aa, Homo sapiens
VDIPEN

SEQ ID NO: 13 - protein, 5 aa, Homo sapiens
FFGVG

SEQ ID NO: 14 - protein, 7 aa, Homo sapiens
ARGSVIL

SEQ ID NO: 15 - protein, 6 aa, Homo sapiens
ARGSVI

SEQ ID NO: 16 - protein, 5 aa, Homo sapiens
FFGVG

SEQ ID NO: 17 - protein, 5 aa, Homo sapiens
ARGSV

SEQ ID NO: 18 - protein, 6 aa, Homo sapiens
NITEGE